(19) United States
(12) Patent Application Publication
(10) Pub. No.: US 2014/0276307 A1
(43) Pub. Date: Sep. 18, 2014

(54) ORTH0PIC BELT WITH MAGNETS

(71) Applicant: William Andrew Landtbom, San Francisco, CA (US)

(72) Inventor: William Andrew Landtbom, San Francisco, CA (US)

(21) Appl. No.: 14/204,492

(22) Filed: Mar. 11, 2014

Related U.S. Application Data
(60) Provisional application No. 61/786,407, filed on Mar. 15, 2013.

(51) Int. Cl. A61F 5/02

(52) U.S. Cl. CPC A61F 5/028
USPC 602/19

(57) ABSTRACT

An orthopedic pad for wearing about the lumbar region of a user and comprising a smoothly contoured body having a template surface bounded by an upper margin and a lower margin. The orthopedic pad includes a central trough portion extending between said upper and lower margins for accommodating the spinal processes of a wearer when the orthopedic pad is pressed against the lumbar region. The orthopedic pad includes two raised plateau regions flanking the central trough portion for contacting the erector spinae muscles of the wearer to provide support therefor. The central trough and the plateau form a smoothly curving surface approximating the lordotic curve of a wearer to assist maintaining spinal posture. The orthopedic pad includes a magnetic pad formed of one or more magnets containing one or more concentric circle partitions conforming to the contoured template surface and extending between the margins and extending over the central trough and the two raised plateau regions to provide a therapeutic magnetic field to the lumbar region.

### BACKGROUND OF THE INVENTION

This invention relates to orthopedic devices for providing support, pain relief or injury prevention for the lower back. More particularly, this invention relates to an orthopedic device including a belt with magnets adapted to be worn by a user to provide support, pain relief or injury prevention for the user.

The cause of back injuries and pain can be stated: “It's not WHAT you lift, but HOW you lift, and HOW you do everything else”. Only 4% of back injuries are “one-event” situations, where the cause is easily definable. The rest involve a level of repetitive motion damage, exacerbated by poor postural transitions, stretching spinal tissues, degrading them until they reach the “ready-to-go” stage. A simple sneeze or tying one's shoes can be the final straw, but these injuries are normally termed as “lifting” injuries.

Many orthopedic devices in the form of belts have been proposed to provide lower back support to avoid injuries and to prevent or alleviate lower back pain. Belts designed for this purpose have generally been of two different types. A first type uses a conforming pad, typically made of foam material, with the pad being permanently attached to the belt, either inside or outside the fabric of the belt, with the pad in a position to be compressed against the lower back region when the belt is arranged about the waist of the user. The second type employs an inflatable air bladder having one or more air chambers with the wall of the bladder being sufficiently flexible to enable the air chamber to conform to the shape of the back of the wearer when the belt is attached around the waist.

Both types of belt devices suffer from the disadvantage that the portion of the device in contact with the back of the user (the pad or the air chamber walls) functions to easily conform to the shape of the wearer's back. Consequently, if the lower back is in a position other than for ideal posture, the belt does not promote a change to the proper posture since the contact region of the pad adapts to the shape of the lower back. When this shape is contorted, as for example by performing a stressful lifting routine, the back is not effectively impeded by the device from maneuvering to an improper posture.

There have been many variations of back belts over the years. For example, elastic back belts have been available with and without suspenders. The belts are wrapped tightly around the waist and cinched with Velcro or similar closures. These devices made several safety claims, such as “This product will conform to the wearer's body, for comfort” or “This product should be tightened during any lifting activities, but loosened and allowed to hang untightened by the suspenders during non-lifting periods”. If metal stays were present, the claim was often made that “The metal stays will remind the wearer to lift properly”. While many belt devices have been and are available, they do not properly or adequately provide support and improvement of spinal mechanics or relieve or prevent pain. These devices conform to the body and do not provide a template to conform the spine.

A dramatic improvement in orthopedic belts has been provided in the devices described in U.S. Pat. No. 5,429,587 entitled ORTHOPEDIC PAD and U.S. Pat. No. 5,651,763 entitled ORTHOPEDIC BELT. These patents describe ortopedic devices that provide a lumbosacral support system comprising a belt and an orthopedic pad carried by the belt. The orthopedic pad has a contoured template surface with a transversely extending central trough portion for accommodating the protruding spinal processes of the wearer when the pad is pressed against the lumbar region, and a pair of raised plateau regions flanking the central trough portion for contacting the erector spinae muscles of the wearer to provide support and improvement of spinal mechanics. The template surface of the pad has a vertical surface contour through the central region approximating the average lordotic curve of a wearer. The belt is longitudinally tapered so the pad is arranged at an angle to vertical to optimally engage the lumbar region of the wearer.

The devices described in U.S. Pat. Nos. 5,429,587 and 5,651,763 provide a firm, curved surface, pressed against the spine that tends to assist spinal tissues to maintain correct spinal posture by conforming to the surface of the curved, firm lumbar pad. Later, a Harvard Med School paper found that a “curved, firm surface” created “proprioceptive feedback”, a subconscious effect where the spine would conform to a predetermined lumbar pad, improving spinal mechanics. This dynamic occurs where spinal tissues act independently of the conscious mind, to reorient their positioning in order to stay within a safe range of motion. Because poor spinal mechanics becomes an ingrained habit, change is very difficult to achieve at the conscious level. Additionally, two peer reviewed studies found that this lumbar pad did maintain a safe range of motion in a simple reaching task (no lifting, where the conscious mind would take over) and credited the result to “proprioceptive feedback”. Finally, the American Osteopathic Academy of Sports Medicine, in granting Back-A-Line, the owner of U.S. Pat. Nos. 5,429,587 and 5,651,763, the only Seal of Acceptance granted in their history, also praised the curved, firm lumbar pad as the reason.

Magnets have also been proposed for relieving pain in the lower back region. Magnetic therapy is intended to relieve pain by increasing blood flow to shrink swollen tissues that are causing the pain. Magnetic therapy to be effective requires magnets that adequately deliver an effective magnetic field of sufficient strength to the tissue to be affected. There are many different types of magnets and many of them have no significant therapeutic effect.

Spinal weakening or damage occurring because of poor spinal mechanics often result in chronic back pain due to swollen or stretched tissues and insufficient blood flow. Even if back pain is treated with magnetic therapy, however, such treatment does not improve spinal mechanics nor eliminate the risk of future spinal damage. The prior spinal mechanics may have resulted in compressed disks, stretched and weakened ligaments and other problems that would result in (current) “residual pain” and would continue to deteriorate further without the introduction of improvements to the spinal mechanics.

Existing “residual pain”, caused by prior poor spinal mechanics or other problems, when treated by magnetic therapy will not improve significantly unless the magnets are of sufficient strength and characteristics to access the lumbar region and the affected spinal tissues. Such “residual pain” may also lead to undesirable compensating spinal mechanics as a means of lessening the existing pain, but such compensating spinal mechanics may cause other negative effects on the tissues that do the compensating.

In consideration of the above background, there is a need for improved orthopedic belts that improve spinal mechanics, reduce pain in the lumbar region and provide relief for chronic back pain from prior spinal damage.

### SUMMARY

The present invention is an orthopedic pad for wearing about the lumbar region of a user and comprising a smoothly contoured body having a template surface bounded by an upper margin and a lower margin. The orthopedic pad includes a central trough portion extending between said upper and lower margins for accommodating the spinal processes of a wearer when the orthopedic pad is pressed against the lumbar region. The orthopedic pad includes two raised plateau regions flanking the central trough portion for contacting the erector spinae muscles of the wearer to provide support therefor. The central trough and the plateau form a smoothly curving surface approximating the lordotic curve of a wearer to assist maintaining spinal posture. The orthopedic pad includes a magnetic pad conforming to the contoured template surface and extending between the margins and extending over the central trough and the two raised plateau regions to provide a therapeutic magnetic field to the lumbar region.

In one embodiment the magnetic pad is formed of one or more concentric circle magnets.

The foregoing and other objects, features and advantages of the invention will be apparent from the following detailed description in conjunction with the drawings.

### BRIEF DESCRIPTION OF THE DRAWINGS

FIG. 1 is a perspective view illustrating an embodiment of an orthopedic belt partially closed.

FIG. 2 is a section view taken along lines 2-2′ of FIG. 1 and illustrating the pad to be placed against the lower back region.

FIG. 3 is a front view of the template surface of the orthopedic pad portion of the belt of FIG. 1.

FIG. 4 depicts a back view of a person's torso wearing the belt of FIG. 1.

FIG. 5 depicts a side view of a person's torso wearing the belt of FIG. 1.

FIG. 6 depicts a side view of a person's torso wearing the belt of FIG. 1 and showing how it aligns with the lumbar region of the person's spine.

FIG. 7 depicts a top view of one of the magnets used in the belt of FIG. 1.

FIG. 8 depicts a front view of the magnet of FIG. 7.

FIG. 9 depicts an expanded and exploded front view of the magnet of FIG. 8.

FIG. 10 depicts a pair of the magnets of the FIG. xs9 type where the pair includes an NSNSNSN magnet and an SNSNSNS magnet.

### DETAILED DESCRIPTION

In FIG. 1, a perspective view is shown illustrating a first embodiment of a belt 10 partially closed. The belt 10 has overlapping ends 11 and 12 and has a central region generally designated with numeral 14. In region 14, the edges of the belt curve outwardly to a maximum width dimension at essentially the center of region 12. The two overlapping ends 11 and 12 are provided with complementary hook and eye fasteners in order to provide an adjustable closure for the belt. A secondary closure is provided by additional belting 16 and 17 of reduced width. The belt 10 can be fabricated from any suitable material, such as a Nylon fabric, and is preferably a two-ply belt secured around the perimeter so as to enable the retention there of an orthopedic pad hereinafter described (see FIG. 3).

In FIG. 2, a section view taken along lines 2-2′ of FIG. 1 is shown illustrating the back portion 14 of the belt 10 of FIG. 1 to be placed against the lower back region of a person wearing the belt. The back portion 14 includes a magnetic pad 20 attached as part of the top surface of the back portion 14.

In FIG. 2, the magnetic pad 20 has a contoured template surface which conforms to the contoured template surface of the back portion 14. The contoured template surface of the magnetic pad 20 and back portion 14 have a transversely extending, normal to the plane of the drawing page, central trough portion 20-1 for accommodating the protruding spinal processes of the wearer when the magnetic pad 20 is pressed against the lumbar region, and a pair of raised plateau regions 20-1 and 20-2 flanking the central trough portion 20-1 for contacting the erector spinae muscles of the wearer to provide support.

In FIG. 3, a front view is shown of the template surface of the magnetic pad 20 and back portion 14 portion of the belt 10 of FIG. 1. The magnetic pad 20 is glued or otherwise attached to back portion 14 and hence attaches the internal magnets 51 and 52 along the trough 20-1 and extending upwards over the raised plateau regions 20-1 and 20-2.

In FIG. 4, a back view of a person's torso 50 is shown wearing the belt 10 of FIG. 1. The region 14 has a back side that faces outward with the magnets 51 and 52 of FIG. 3 facing inward against the lower back portion of the torso 50.

In FIG. 5, a side view of a person's torso 50 is shown wearing the belt 10 of FIG. 1. The region 14 has a back side that faces outward with the magnets 51 and 52 of FIG. 3 facing inward against the lower back portion of the torso 50. The belt 10 is positioned about the waist of the user's torso 50 at an angle 21, which typically is about 7.degrees. The template surface including the trough 20-1 and the raised plateau regions 20-1 and 20-2 of the magnetic pad 20 rests against the wearer's back in the lower back region. The plateau regions 20-1 and 20-2 make surface contact with the erector spinae muscles of the wearer through the skin, and the central hollow trough 20-1 accommodates the bony protrusions of the spine. The magnetic pad 20 responds to extension of the back away from the idealized posture represented by the template surface of magnetic pad 20 by opposing such extension away from the idealized posture in response to the relative stiffness of the magnetic pad 20 material. The lower back has a natural tendency to adapt to and follow the smooth surface of the magnetic pad 20 which guides the lower back into ideal posture.

In FIG. 6, a side view of a person's torso wearing the belt of FIG. 1 is shown, with the same view as in FIG. 5, and additionally showing how the belt 10 and the magnetic pad 20 align with the lumbar region and the spine of the torso 50. The torso 50 includes the thoracic region with twelve vertebrae and the lumbar region with five vertebrae. The location and orientation of the belt 10 is shown by a dotted line superimposed over the skeleton portion of the lumbar region. The belt 10 is positioned about the waist of the user's torso 50 at an angle 21, which typically is about 7 degrees which generally matches the angle of the lumbar region 14′ of the user's torso. The vertebrae 60 are separated by disks 61. The lumbar region 14′ of the user's torso additionally includes muscles, tendons, skin and other body components not shown but which contribute to the movement and function of the lumbar region and which can suffer from poor spinal mechanics.

In FIG. 7, a top view of the magnet 51 is shown and is representative of one of the magnets used in the belt 10 of FIG. 1. The magnet 51 is a Concentric Circle Magnet of the type available from BIOflex Medical Magnets, Inc. and of the type described in U.S. Pat. No. 7,611,453 entitled APPARATUS AND METHOD FOR STATIC MAGNETIC FIELD TREATMENT OF TISSUE, ORGANS, CELLS, AND MOLECULES.

The magnet 51 in the top view is circular and includes the concentric circular regions 55-1, 55-2, 55-3 and 55-4 which have polarities, indicated by north (N) and south (S), of SNSNSNS when viewed across a major axis such as section line 8-8′. The concentric circular regions (partitions) 55-1, 55-2, 55-3 and 55-4 have diameters of 24 mm, 46 mm, 68 mm and 90 mm, respectively, in the embodiment of FIG. 7. The diameters given are not critical and can vary widely. In general, the diameters correlate to the depth of penetration of the magnetic field into the lumbar region of a body.

In FIG. 8, a front view is shown of the magnet 51 of FIG. 7. The magnet 51 includes an upper layer 51-1 formed of concentric rings and includes a bottom layer 51-2 formed of a single axial magnet. In the embodiment of FIG. 8, the magnet 51 has a thickness of approximately 1.5 millimeters (mm). The thickness for producing magnets in general is not critical and can vary widely. The thickness for use in the orthopedic belt 10 needs to be thin enough to permit the magnetic pad 20 to conform to the curvature of the trough 20-1 and the raised plateau regions 20-1 and 20-2 as discussed in connection with FIG. 2 and FIG. 3.

In FIG. 9, an expanded and exploded front view is shown of the magnet 51 of FIG. 8. The magnet 51 includes an upper layer 51-1 formed by concentric rings and includes a bottom layer 51-2 formed by a single axial magnet. The upper layer 51-1 includes cross sections of the concentric circular regions 55-1, 55-2, 55-3 and 55-4 of FIG. 7. In FIG. 9, the ring sections 55-1 and 55-3 have a North-South (NS) orientation where North (N) is toward the top of the drawing page and South (S) is toward the bottom of the drawing page. The ring sections 55-2 and 55-4 have a South-North (SN) orientation. The bottom layer 51-2 is formed as a single axial magnet having a North-South (NS) orientation where North (N) is toward the top of the drawing page and South (S) is toward the bottom of the drawing page. If viewed from the top, the layer 51-2 would appear as a continuous 90 mm circle with the entire top having a North polarization. If viewed from the bottom, the layer 51-2 would appear as a continuous 90 mm circle with the entire bottom having a South polarization.

In FIG. 10, two magnets 51 and 52 of the FIG. 9 type are shown. The magnet 51 includes the concentric circle polarity orientation NSNSNSN and the magnet 52 includes the opposite concentric circle polarity orientation SNSNSNS. The magnet 52 is formed by replacing all the N's with S's and all the S's with N's in FIG. 7, FIG. 8 and FIG. 9. As shown in FIG. 10, the concentric circle partitions 56-1, 56-2, 56-3 and 56-4 for magnet 52 are of the opposite polarity of the concentric circle partitions 55-1, 55-2, 55-3 and 55-4 for magnet 51. The magnets 51 and 52 are arranged in the magnetic pad 20 which is more than 189 mm high by 90 mm wide. The thickness of the pad 8, as indicated by the magnet thickness in FIG. 8, is approximately 1.5 mm.

In one preferred embodiment, the magnetic pad 20 is a flexible pad made from polycarbonate or other binding material. In manufacture, the binding material is heated and mixed with ferrite powder and/or other magnetic powders. The mixture is rolled into a thin flexible sheet and cooled to form a magnetizable sheet. Magnetic fields are applied to the magnetizable sheet to form the concentric circle partitions 56-1, 56-2, 56-3 and 56-4 for magnet 52 and the concentric circle partitions 55-1, 55-2, 55-3 and 55-4 for magnet 51. The magnetic powers and magnetization steps provide the gauss strength and penetration characteristics which permit the magnetic fields from the magnets 51 and 52 to penetrate the lumbar region and reach tissues and surrounding regions that can benefit from the magnetic field produced.

It is apparent from the above description that an orthopedic belt 10 for wearing about the lumbar region of a user is described. The belt 10 has a smoothly contoured body, including a magnetic pad 20, with a template surface 15 bounded by an upper margin 18 and a lower margin 19. The belt 10 and the magnetic pad 20 include a central trough portion 20-1 extending between the upper margin 18 and the lower margin 19 for accommodating the spinal processes of a wearer when the magnetic pad 20 is pressed by belt 10 against the lumbar region. The belt 10 and the magnetic pad 20 include two raised plateau regions 20-2 and 20-3 flanking the central trough portion 20-1 for contacting the erector spinae muscles of the wearer to provide support therefor. The central trough 20-1 and the plateau regions 20-2 and 20-3 form a smoothly curving surface 15 approximating the lordotic curve of a wearer to assist maintaining spinal posture. The orthopedic belt 10 includes a magnetic pad 20 conforming to the contoured template surface 15 and extending between the margins 18 and 19 and extending over the central trough 20-1 and the two raised plateau regions 20-2 and 20-3 to provide a therapeutic magnetic field to the lumbar region. In the embodiment described, the magnetic pad is formed of one or more concentric circle magnets such as concentric circle magnets 51 and 52 containing concentric circle partitions 55 and 56.

While the invention has been particularly shown and described with reference to preferred embodiments thereof it will be understood by those skilled in the art that various changes in form and details may be made therein without departing from the scope of the invention.

1. An orthopedic belt for wearing about the lumbar region of a user comprising,
a smoothly contoured body having a template surface bounded by an upper margin and a lower margin including,
a central trough portion extending between the upper and lower margins for accommodating the spinal processes of a wearer when the pad is pressed against the lumbar region,
two raised plateau regions flanking said central trough portion for contacting the erector spinae muscles of the wearer to provide support therefor,
the central trough and the plateau forming a smoothly curving surface approximating the lordotic curve of a wearer to assist maintaining spinal posture,
a magnetic pad conforming to the contoured template surface and extending between the margins and extending over the central trough and the two raised plateau regions to provide a therapeutic magnetic field to the lumbar region.

2. The pad of claim 1 wherein the magnetic pad is formed of one or more concentric circle magnets containing concentric circle partitions.
